(19) United States
(12) Patent Application Publication
(10) Pub. No.: US 2002/0042127 A1
(43) Pub. Date: Apr. 11, 2002

(54) DONOR CELLS EXPRESSING FUSOGENS

(76) Inventors: STEPHEN JAMES RUSSELL, ROCHESTER, MN (US); RICHARD VILE, ROCHESTER, MN (US); ANDREW BATEMAN, ROCHESTER, MN (US); ALAN MELCHER, ROCHESTER, MN (US)

Correspondence Address: FISH & RICHARDSON P.C., P.A., 60 SOUTH SIXTH STREET, SUITE 3300, Minneapolis, MN, 55402, US

(21) Appl. No.: 09/444,802

(22) Filed: Nov. 22, 1999

(30) Foreign Application Priority Data
Nov. 20, 1998 (GB) 982542.5

(51) Int. Cl. A61K 48/00; A01N 63/00; C12N 5/00; C12N 5/06; C12N 5/12; C12N 15/02

(52) U.S. Cl. 435/346; 435/449

(57) ABSTRACT

Disclosed is a cell which expresses a surface marker associated with a professional antigen presenting cell, and a fusogenic membrane protein, where the cell may also express at its surface a tumor cell marker. Also disclosed is a fusion hybrid formed by the fusion of a tumor cell and a professional antigen presenting cell (APC) such that the resulting fusion hybrid expresses an APC marker, a tumor cell marker, and a fusogenic membrane glycoprotein. Also disclosed are compositions comprising the cells and fusion hybrids, and methods of making and using the hybrids.

### BACKGROUND OF THE INVENTION

Cell-cell fusion occurs naturally in some cell types, or in cells infected with any of a number of viruses encoding fusogenic proteins, or following chemical treatment of cells. Recruitment of cells into syncytia, large multinucleate agglomerations of fused cells, results in the death of the fused cells.

Fusogenic membrane glycoproteins (FMGs) have been found to induce syncytium formation when expressed in isolation from the remainder of the virus. International patent application No. WO98/40492 discloses a recombinant nucleic acid expression vectorencoding a fusogenic membrane polypeptide from a virus, and a method of treating malignant disease, by administering to a patient the recombinant nucleic acid vector, where the vector is taken up by cancerous cells in the patient and thus causes the cancer cells to fuse and die.

Antigen presenting cells (APCs) are cells specialized for the ability to process and present peptide antigens in a form such that the antigens are recognized by T cells, thereby stimulating the development of activated T cells specific for those antigens. Fusions of tumor cells with APCs have previously been achieved using physical methods to induce the fusions ex vivo (Guo et al Science 1994 263:518-520). In addition, gene transfer of antigen-presenting molecules to tumor cells to stimulate tumor antigen presenting capacity was shown by Tanaka et al. (1988, Molecular and Cellular Biology 8:1857-1861). It has also been demonstrated that tumor cells traffic to, and into, tumor deposits through experiments using suicide gene therapies based on Herpes Simplex Virus (HSV) tk (thymidine kinase) (Freeman et al., Human Gene Therapy 1995 6:927-939). That is, re-introduced tumor cells tend to localize to and infiltrate the original tumor.

There is a need in the art for improved methods of killing tumor cells. In particular, there is a need in the art for methods that not only kill tumor cells directly, but also stimulate a patient's own immune defenses to kill tumor cells.

### SUMMARY OF THE INVENTION

The invention encompasses an isolated cell expressing on its surface a fusogenic membrane protein and a professional antigen presenting cell marker.

Preferably, the isolated cell further comprises on its surface a tumor cell marker.

In other preferred embodiments,the fusogenic membrane protein is a viral fusogenic membrane glycoprotein.

The invention also encompasses a hybrid cell comprising a tumor cell fused to a professional antigen presenting cell.

Preferably, the tumor cell and/or the antigen presenting cell is obtained from a patient to whom the cell is to be administered.

Preferably, the tumor antigen is an antigen which is expressed in a tumor cell line.

The invention also encompasses a method of making a hybrid cell, the method comprising contacting a tumor cell with a professional antigen presenting cell under conditions which permit cell-cell fusion, wherein one of the tumor cell or the profesional antigen presenting cell expresses a fusogenic membrane protein receptor.

Preferably, the contacting step is performed in vitro or ex vivo, or the contacting step is performed in situ in a patient.

The invention also encompasses a method of preparing a therapeutic composition for the treatment of malignant disease, the method comprising the step of admixing a cell as described above with a physiologically acceptable carrier.

The invention also encompasses a therapeutic composition comprising a cell described herein, in admixture with a physiologically acceptable carrier.

The invention also encompasses a method of treating a malignant disease in a mammal, the method comprising the step of administering a cell according to the invention to the mammal in an amount effective to reduce a symptom of the malignant disease.

Preferably, the method further comprises, prior to administering, the step of fusing a tumor cell with a professional antigen presenting cell in vitro or ex vivo to form the cell.

The invention also encompasses a method of treating a malignant disease in a mammal, the method comprising the step of administering a professional antigen presenting cell expressing a fusogenic membrane protein to the mammal in an amount effective to reduce a symptom of the malignant disease.

The invention also encompasses a method of treating a malignant disease in a mammal, comprising administering to the mammal an autologous tumor cell suspension expressing on its surface a fusogenic membrane protein in an amount effective to reduce a symptom of the disease.

The invention also encompasses a method of vaccinating a mammal against a malignant disease, comprising administering to the mammal a cell expressing on its surface a fusogenic membrane protein, a professional antigen presenting cell marker and a tumor cell marker in an amount effective to elicit an increase in the number of T cells specific for the tumor cell marker.

The invention also encompasses a method of vaccinating a mammal against a malignant disease, comprising administering to the mammal an autologous tumor cell expressing on its surface a fusogenic membrane protein and a tumor cell marker in an amount effective to elicit an increase in the number of T cells specific for the tumor cell marker.

As used herein, the term “isolated cell” refers to a cell that is removed from its natural environment and is substantially pure. For example, APCs may be isolated from a sample of the patient's peripheral blood; dendritic cells are isolated from bone marrow or from spleen tissue; macrophages may be isolated from peripheral blood, or, alternatively, from alveolar lavage fluid or from peritoneal lavage fluid; tumor cells may be isolated from a tissue biopsy sample from a diseased individual, for example, primary fibroblasts are isolated, for example, from a skin biopsy. “Substantially pure” means that the isolated cell, if it occurs in a mixture of other cells, constitutes at least 50%, and preferably 75%, 80%, 90%, and 95% of the cells in the mixture. “Pure” means that the isolated cell is the only cell type in a preparation.

As used herein, the term “hybrid cell” or “fusion hybrid” refers to a cell formed when two or more cells are made to fuse together. As used herein, a hybrid cell is a hybrid of a tumor cell and an antigen presenting cell, such that the hybrid expresses on its surface one or more markers associated with a professional antigen presenting cell, one or more tumor antigens that define a tumor cell type, and a fusogenic membrane protein. Thus, a hybrid cell according to the invention is distinguished from prior art cell hybrids by the presence of three distinct components on its cell surface, an APC marker, a tumor cell marker, and a fusogenic membrane protein.

As used herein, the term “tumor cell” refers to a transformed cell of an individual or to a cell from a transformed cell line. The term encompasses a cell of a “tumor cell vaccine line”, which is a tumor cell line that expresses one or more tumor cell antigens and is capable of eliciting an anti-tumor immune response following administration of irradiated cells of the tumor cell line to a patient. Characteristics of tumor cells include anomalous behavior in tissue culture (for example, growth factor independence, loss of contact inhibition, capacity for anchorage-independent growth, growth to higher density than non-tumor cells, and failure to reach senescence after multiple passages), the ability to invade tissues or metastasize to distant sites, the ability to form tumors when injected into nude mice, and the ability to stimulate anglogenesis.

As used herein, the term “antigen” refers to a peptide or polypeptide that elicits an immune response in a mammal (i.e., at least a T cell response). An antigen may be “self” (i.e., a polypeptide that is made by the mammal and present ina helathy individual, or “non-self”, that is, a polypeptide that is not normally present in a healthy individual (i.e., a foreign polypeptide).

As used herein, the term “tumor cell marker” (“tumor antigen” or “tumor associated antigen”) refers to a class of protein markers, or antigens that tend to be expressed to a greater extent in transformed tumor cells than in non-transformed cells. As such, tumor antigens may be expressed by non-tumor cells, although usually at lower concentrations or during an earlier developmental stage of a tissue or organism. Tumor cell markers are disclosed hereinbelow.

As used herein, the term “professional antigen presenting cell” refers to a cell that has the cellular mechanisms necessary to present on its surface an antigen (that is, a non-APC polypeptide, one which is not normally present on the surface of an APC) and one or more major histocompatibility complex Class II antigens. A cell with the cellular mechanisms necessary to present antigen can ingest antigen in a receptor-mediated manner and re-present on its surface peptides derived from the antigen in combination with MHC Class I or II antigens. Examples of naturally-occurring professional APCs include macrophages, dendritic cells and B lymphocytes. Macrophages express at least the following combination of cell surface “markers”: CD11a, -b, and -c, CD16, CD17, CD63, CD64, CD68 and CD71. Dendritic cells express at least the following combination of cell surface “markers”: CMRF-44, CD83 and CMRF-56. B lymphocytes express at least the following combination of cell surface “markers”: CD19, CD20, CD21, CD22, CD40, CD72, and CD78.

As used herein, the term “surface markers associated with a professional antigen presenting cell” refers to MHC Class I and II markers, F_{c} receptors, and adhesion molecules. MHC Class I markers include HLA (human leukocyte antigen) -A, -B, and -C, and MHC Class II markers include HLA-DR, -DQ, and -DP. Fc receptors include, but are not limited to CD16 (FcRIII), CD23 (FcεRIIb), CDw32 (FcRII), and CD64 (FcRI). Adhesion molecules broadly expressed by antigen presenting cells include, but are not limited to CD11a (LFA-1α), CD18 (LFA-1β), CD29 (VLA-β), CD54 (ICAM-1), and CD58 (LFA-3). A given APC (macrophage, dendritic cell, B cell or synthetic APC) according to the invention expresses at least one MHC Class II marker on its surface and may express one or more of the MHC Class I, Fc receptor or adhesion molecules.

As used herein, the term “syncytium inducing polypeptide” refers to a polypeptide or a portion thereof that induces cell-cell fusion resulting in formation of a syncytium.

The term “syncytium” refers to a cell-cell fusion which appears in a tissue biopsy or tissue culture sample as a large acellular area with multiple nuclei, i.e., a multinucleate region of cytoplasm.

A “fusogenic membrane protein” is a “syncytium inducing polypeptide,” which is a polypeptide that, when expressed on the surface of a cell, mediates the fusion of that cell with another cell expressing a cell surface receptor for that polypeptide. A “fusogenic membrane glycoprotein” as used herein has the ability to mediate or induce fusion between a cell expressing the fusogenic membrane glycoprotein and a cell expressing a receptor for the fusogenic membrane glycoprotein. Examples of fusogenic membrane proteins include, but are not limited to fertilin β, and fusogenic membrane glycoproteins, including viral fusogenic membrane glycoproteins or recombinant forms thereof modified to be selective for a given cell surface receptor or to have enhanced fusogenicity.

A “viral fusogenic membrane glycoprotein” is a virally-derived fusogenic membrane protein that, in nature, mediates membrane fusion of a virus to its host target cell. The viral fusogenic membrane glycoprotein subset of the fusogenic membrane proteins includes, but is not limited to: type G glycoproteins in Rabies, Mokola, vesicular stomatitis and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus, canine distemper virus, Newcastle disease virus, human parainfluenza virus 3 , simian virus 41, Sendai virus and human respiratory syncytial virus; gH of human herpesvirus 1 and simian varicella virus, with the charepone protein gL; human, bovine and cercopithicine herpesvirus gB; envelope glycoproteins of Friend murine leukemia virus and Mason Pfizer monkey virus; influenza haemagglutinin; G protein of Vesicular Stomatitis Virus; mumps virus hemagglutinin neuraminidase, and glycoproteins F1 and F2; and membrane glycoproteins from Venezuelan equine encephalomyelitis.

Virus-cell fusion and cell-cell fusion are distinct processes. “Fusogenic” refers to the biological activity of a viral membrane glycoprotein to promote virus-cell fusion when in its natural virus context. A “fusogenic effect” refers to the natural biological activity of a fusogenic polypeptide in inducing cell fusion via the presence of a virus encoding and expressing the fusogenic polypeptide. In contrast, “syncytium-induction” refers to the biological activity of a syncytium-inducing polypeptide, which may be a viral membrane glycoprotein substantially isolated from its natural virus context, to induce cell-cell fusion without the virus. To be useful according to the invention, a viral glycoprotein which has a fusogenic effect when carried in the virus must be capable of inducing syncytium formation when in substantial isolation from the virus. “Substantial isolation” from the virus means that a given fusogenic viral membrane glycoprotein preparation is not contaminated with (contains no more than 5% by weight) other proteins encoded by the virus, including viral capsid proteins and transcription or replication factors.

As used herein, the term “syncytium inducing polypeptide receptor” or “fusogenic membrane protein receptor” refers to a cell surface polypeptide or other molecule that serves as the receptor for a given syncytium inducing polypeptide or fusogenic membrane protein. Viral fusogenic membrane glycoprotein receptors are reviewed by Weiss & Tailor (1995, Cell 82: 531-533). Examples of viral fusogenic membrane protein receptors include CD46 (receptor for measles virus F and H fusion proteins), PIT-1 (receptor for Gibbon age leukemia virus), CD4 (receptor for HIV), CAT (receptor for MLV (murine leukemia virus) -E), and Ram-1 (receptor for MLV-A). Receptors for non-viral fusogenic membrane proteins include, for example, alpha-6, beta-1 integrin, the receptor for fertilin β(GenBank Accession No. X69902).

As used herein in reference to cell-cell fusion, or making a cell-cell hybrid, the term “in vitro” or “ex vivo” means that the hybrid cell is made outside the body of the individual to whom it is to be administered. “Ex vivo” means that a cell is removed from an individual and is treated “in vitro”. Thus, the cell may be re-introduced into the individual. Also in this context, the terms “in situ” or “in vivo” mean that the hybrid cell forms within the body of the individual, after administration of fusogenic membrane protein-expressing cells. “In situ” means that administration is performed at the site where syncytium induction occurs; “in vivo” means that hybrid cells may be present both at the tumor site in the patient and also carried in the bloodstream.

As used herein, the term “malignant disease” refers to a disorder or symptoms resulting from the proliferation of oncogenically transformed cells. Symptoms of malignant disease vary depending upon the nature of the transformed cell type and the particular location(s) of tumors or transformed cells. For example, bronchogenic carcinoma, or lung cancer, of which there are several forms, including squamous cell carcinoma, adenocarcinoma, small cell carcinoma and large cell carcinoma, has the symptoms of cough, dyspnea, chest pain, hemoptysis and anorexia, in addition to the presence of a tumor mass on X-ray. As another example, acute leukemia often has the symptoms of weakness, malaise, anorexia, bone and joint pain, fever, petechiae, lymph node swelling, and splenomegaly, in addition to the presence of abnormal cells in the peripheral blood. As another example, brain tumors often have the symptoms of personality changes, intellectual decline, emotional lability, seizures, headaches, nausea. Depending upon the site of the tumor mass, brain tumors may cause visual field defects, hearing loss, loss of or altered olfactory function, motor phenomena and aphasia, among other symptoms.

As used herein, the term “reduce symptoms” refers to a decrease in the severity or extent of the indicators of a disease. Examples of specific symptoms that may be directly quantitated include fever, high (or low) white blood cell count, elevated or decreased blood pressure, or the presence of abnormal cells in a blood or tissue specimen. A change of 1% or more, 2% or more, 5% or more, 10% or more, up to 25%, 50% or 75% or more in these or other quantitative measurements of disease status is considered to be indicative of reduced symptoms. Other symptoms such as pain, lethargy, nausea and restlessness, among others, may be considered reduced if there is a difference noted by the physician or reported by the patient following treatment, and the difference persists over time, for example, for two days, for two weeks, for two months, or longer.

As used herein in reference to tumor size or disease symptoms, the term “maintain” means that the size or symptoms do not increase or worsen in severity with the passage of time, for example, over weeks or months.

As used herein, the term “therapeutic composition” refers to a composition effective for the treatment of a disease.

As used herein, the term “physiologically acceptable carrier” or “diluent” refers to a solution or composition in which cells or hybrid cells of the invention may be suspended to allow administration (e.g., intravenously, intraperitoneally, etc.) of the hybrids or cells to an individual. A physiologically acceptable carrier or diluent will generally be isotonic and will often be buffered; a large number of acceptable diluents or carriers are known in the art.

As used herein, the term “administering” refers to the introduction of cells or hybrids of the invention to an individual for therapeutic purposes. As noted above, cells or hybrids may be administered, for example, intravenously, intraperitoneally, or even directly into a tumor.

As used herein, the term “conditions which permit fusion” means that a cell expressing a fusogenic membrane protein and a cell, either engineered to express or naturally expressing a corresponding fusogenic membrane protein receptor, are mixed together at a concentration and ratio such that contact between the fusogenic membrane protein on one cell and the other cell occurs, resulting in fusion of the two cells, such that at least one cell surface protein on each cell is expressed in the resulting fusion hybrid in addition to the fusogenic membrane protein. A concentration of 10³ to 10⁸ cells per ml is acceptable, and a ratio of fusogenic membrane-protein-expressing cell to fusion partner in the range of 1:100, 1:10, 1:1, 10:1, or 100:1 is acceptable for conditions that permit cell fusion.

As used herein, the term “amount effective to reduce the symptoms” means that a number of hybrid cells (formed in vitro or ex vivo; in the range of 10³-10⁸ cells, as described herein) or a number of APCs expressing an FMP (in the range of 10³-10⁸ cells, as described herein) is administered to a patient in need of treatment for a tumor such that the outward indicators of the disease caused by the tumor are decreased.

As used herein, the term “autologous” means that cells or materials are derived from the individual to whom they are to be administered.

As used herein, the term “allogeneic” means that cells or materials are derived from the same species as the individual to whom they are to be administered.

As used herein, the term “xenogeneic” means that cells or materials are derived from a different species than the individual to whom they are to be administered.

### DESCRIPTION

The invention relates to a hybrid cell formed by the fusion of a tumor cell with one or more non-tumor cell types. In this aspect, the invention exploits characteristics of the non-tumor cell type or types in order to enhance the anti-tumor effect of the induced cell-cell fusion. Tumor cells tend to express tumor-associated antigens, or tumor antigens, in high amounts at the tumor cell surface. Antigen-presenting cells (APC) of the immune system are useful as non-tumor fusion partners with tumor cells in that they have the necessary cellular pathways to display tumor cell antigens on their surfaces in combination with major histocompatibility complex (MHC) antigens. Antigens displayed as complexes with MHC proteins are efficiently recognized by the patient's T cells, thereby promoting an anti-tumor immune response to cells expressing the tumor associated antigens. A tumor cell-APC hybrid of the invention expresses not only surface marker(s) associated with a professional APC and a tumor antigen, but also expresses on its surface a syncytium inducing polypeptide that promotes the fusion of the hybrid cell with cells of the tumor, thus allowing further recruitment of fusion partners.

A. Fusogenic Membrane Proteins Useful According to the Invention

Cell-cell fusion according to the invention may be induced by engineering one of the cell types intended to undergo fusion to express a fusogenic membrane polypeptide (FMP). Cells expressing one or more FMPs will serve as fusion donor partners with acceptor target cells. A large number of FMPs are known to those skilled in the art, including FMPs expressed by viruses and by various cell types that naturally undergo cell fusion. The GenBank accession numbers for a number of fusogenic proteins useful according to the invention are provided herein; the amino acid sequences are predicted from the nucleic acid sequences.

1. Virally-derived FMPs.

The FMP will frequently, but not necessarily be a virally-derived polypeptide. Many viruses depend upon fusogenic membrane glycoproteins (which constitute a subset of FMPs) displayed upon their outer surfaces in order to fuse with and enter target cells. These proteins frequently function to induce cell-cell fusion when expressed in isolation from the remainder of the viral genes. Viral fusogenic polypeptide FMGs, both naturally occurring and engineered by recombinant nucleic acid techniques, and suitable for use in the present invention are described in detail in WO 98/40492, the content of which is incorporated herein by reference. WO98/40492 provides recombinant vectors encoding viral FMGs, which vectors are taken up by tumor cells in vivo.

Viral syncytium-inducing polypeptides useful according to the invention include fusogenic membrane glycoproteins which include but are not limited to the following.

a) Membrane Glycoproteins of Enveloped Viruses.

Enveloped viruses have membrane spike glycoproteins for attachment to mammalian cell surfaces and for subsequent triggering of membrane fusion, providing a pathway for viral entry into the cell. In some viruses, attachment and fusion triggering are mediated by a single viral membrane glycoprotein, but in others these functions are provided by two or more separate glycoproteins. Sometimes (e.g. Myxoviridae, Togaviridae, Rhabdoviridae) the fusion triggering mechanism is activated only after the virus has entered into the target cell by endocytosis, at acid pH (i.e., below about pH 6.0). Examples of such membrane glycoproteins in Rhabdoviruses are those of type G in rabies (Genbank Acc. No. U11736), Mokola (Genbank Acc. No. U17064) and vesicular stomatitis (Genbank Acc. Nos. M21417 and J04326) viruses, and those in Togaviruses.

Other viruses (e.g. Paramyxoviridae, Retroviridae, Herpesviridae, Coronaviridae) can fuse directly with the target cell membrane at substantially neutral pH (about 6.0-8.0) and have an associated tendency to trigger membrane fusion between infected target cells and neighboring noninfected cells. The visible outcome of this latter tendency for triggering of cell-cell fusion is the formation of cell syncytia containing up to 100 nuclei. Viral membrane proteins of these latter groups of viruses are of particular interest in the present invention. In addition to those proteins from Paramyxoviruses, Retroviruses and Herpesviruses discussed below, examples of Coronavirus membrane glycoprotein genes include those encoding the murine hepatitis virus JHM surface projection protein (Genbank Acc. Nos. X04797, D00093 and M34437), porcine respiratory coronavirus spike- and membrane glycoproteins (Genbank Acc. No. Z24675) avian infectious bronchitis spike glycoprotein (Genbank Acc. No. X64737) and its precursor (Genbank Acc. No. X02342), and bovine enteric coronavirus spike protein (Genbank Acc. No. D00731).

b) Viral Membrane Glycoproteins of the Paramyxoviridae Viruses.

Viruses of the Family Paramyxoviridae have a strong tendency for syncytium induction which is dependent in most cases on the co-expression of two homo-oligomeric viral membrane glycoproteins, the fusion protein (F) and the viral attachment protein (H, HN or G). Co-expression of these paired membrane glycoproteins in cultured cell lines is required for syncytium induction although there are exceptions to this rule such as SV5 whose F protein alone is sufficient for syncytium induction. F proteins are synthesized initially as polyprotein precursors (F₀) which cannot trigger membrane fusion until they have undergone a cleavage activation. The activating protease cleaves the F₀ precursor into an extraviral F₁ domain and a membrane-anchored F₂ domain which remain covalently associated through disulfide linkage. The activating protease is usually a serine protease and cleavage activation is usually mediated by an intracellular protease in the Golgi compartment during protein transport to the cell surface. Alternatively, where the cleavage signal is not recognized by a Golgi protease, the cleavage activation can be mediated after virus budding has occurred, by a secreted protease (e.g. trypsin or plasmin) in an extracellular location (Ward et al. Virology, 1995, 209, p 242-249; Paterson et al., J. Virol., 1989, 63, 1293-1301).

Examples of Paramyxovirus F genes include those of Measles virus (Genbank Acc. Nos. X05597 or D00090), canine distemper virus (Genbank Acc. No. M21849), Newcastle disease virus (Genbank Acc. No. M21881), human parainfluenza virus 3 (Genbank Acc. Nos. X05303 and D00125), simian virus 41 (Genbank Acc. Nos. X64275 and S46730), Sendai virus (Genbank Acc. No. D11446) and human respiratory syncytial virus (Genbank Acc. No. M11486, which also includes glycoprotein G). Also of interest are Measles virus hemagluttinin (Genbank Acc. No. M81895) and the hemagluttinin neuraminidase genes of simian virus 41 (Genbank Acc. Nos. X64275 or S46730), human parainfluenza virus type 3 (M17641) and Newcastle disease virus (Genbank Acc. No. J03911).

c) Membrane Glycoproteins of the Herpesvirus Family.

Certain members of the Herpesvirdae family are renowned for their potent syncytium-inducing activity. Indeed, Varicella-Zoster Virus has no natural cell-free state in tissue culture and spreads almost exclusively by inducing cell fusion, forming large syncytia which eventually encompass the entire monolayer. gH is a strongly fusogenic glycoprotein which is highly conserved among the herpesvirus; two such proteins are gH of human herpesvirus 1 (Genbank Acc. No. X03896) and simian varicella virus (Genbank Acc. No. U25806). Maturation and membrane expression of gH are dependent on coexpression of the virally encoded chaperone protein gL (Duus et al., Virology, 1995, 210, 429-440). Although gH is not the only fusogenic membrane glycoprotein encoded in the herpesvirus genome (gB has also been shown to induce syncytium formation), it is required for the expression of virus infectivity (Forrester et al., J. Virol., 1992, 66, 341-348). Representative genes encoding gB are found in human (Genbank Acc. No. M14923), bovine (Genbank Acc. No. Z15044) and cercopithecine (Genbank Acc. No. U12388) herpesviruses.

d) Membrane Glycoproteins of Retroviruses.

Retroviruses use a single homo-oligomeric membrane glycoprotein for attachment and fusion triggering. Each subunit in the oligomeric complex is synthesized as a polyprotein precursor which is proteolytically cleaved into membrane-anchored (TM) and extraviral (SU) components which remain associated through covalent or noncovalent interactions. Cleavage activation of the retroviral envelope (env) precursor polypeptide is usually mediated by a Golgi protease during protein transport to the cell surface. There are inhibitory (R) peptides on the cytoplasmic tails of the TM subunits of the envelope glycoproteins of Friend murine leukemia virus (FMLV, EMBL accession number X02794) and Mason Pfizer monkey virus (MPMV; Genbank Acc. No. M12349) which are cleaved by the virally encoded protease after virus budding has occurred. Cleavage of the R peptide is required to activate fully the fusogenic potential of these envelope glycoproteins and mutants lacking the R peptide show greatly enhanced activity in cell fusion assays (Rein et al, J. Virol ., 1994, 68, 1773-1781; Ragheb & Anderson, J. Virol., 1994, 68, 3220-3231; Brody et al, J. Virol. 1994, 68, 4620-4627).

e) MLV (Murine Leukemia Virus) Membrane Glycoproteins with Altered Specificity.

Naturally occurring MLV strains can also differ greatly in their propensity for syncytium induction in specific cell types or tissues. One MLV variant shows a strong tendency to induce the formation of endothelial cell syncytia in cerebral blood vessels, leading to intracerebral hemorrhages and neurologic disease. The altered behavior of this MLV variant can be reproduced by introducing a single point mutation in the env gene of a non-neurovirulent strain of Friend MLV, resulting in a tryptophan-to-glycine substitution at amino acid position 120 in the variable region of the SU glycoprotein (Park et al, J. Virol., 1994, 68, 7516-7524).

f) HIV Membrane Glycoproteins.

HIV strains are also known to differ greatly in their ability to induce the formation of T cell syncytia and these differences are known to be determined in large part by variability between the envelope glycoproteins of different strains. Typical examples are provided by Genbank accessions L15085 (V1 and V2 regions) and U29433 (V3 region).

g) Acid-triggered Fusogenic Glycoproteins having an Altered pH Optimum.

The membrane glycoproteins of viruses that normally trigger fusion at acid pH do not usually promote syncytium formation. However, they can trigger cell-cell fusion under certain circumstances. For example, syncytia have been observed when cells expressing influenza haemagglutinin (Genbank Acc. No. U44483) or the G protein of Vesicular Stomatitis Virus (Genbank Acc. Nos. M21417 and J04326) are exposed to acid (Steinhauer et al, Proc. Natl. Acad. Sci. USA 1996, 93, 12873-12878) or when the fusogenic glycoproteins are expressed at a very high density (Yang et al, Hum. Gene Ther.1995, 6, 1203-1213). In addition, acid-triggered fusogenic viral membrane glycoproteins can be mutated to shift their pH optimum for fusion triggering (Steinhauer et al, Proc. Natl. Acad. Sci. USA 1996, 93, 12873-12878).

h) Membrane Glycoproteins from Poxviruses.

The ability of poxviruses to cause cell fusion at neutral pH correlates strongly with a lack of HA production (Ichihashi & Dales, Virology, 1971, 46, 533-543). Wild type vaccinia virus, an HA-positive orthopoxvirus, does not cause cell fusion at neutral pH, but can be induced to do so by acid pH treatment of infected cells (Gong et al, Virology, 1990, 178, 81-91). In contrast, wild type rabbitpox virus, which lacks a HA gene, causes cell fusion at neutral pH. However, inactivation of the HA or SPI-3 (serpin) genes in HA-positive orthopoxviruses leads to the formation of syncytia by fusion of infected cells at neutral pH (Turner & Moyer, J. Virol. 1992, 66, 2076-2085). Current evidence indicates that the SPI-3 and HA gene products act through a common pathway to control the activity of the orthopoxvirus fusion-triggering viral glycoproteins, thereby preventing fusion of cells infected with wild type virus.

i) Membrane glycoproteins of other replicating viruses.

Replicating viruses are known to encode fusogenic viral membrane glycoproteins, which viruses include but are not limited to mumps virus (hemagglutinin neuraminidase, SwissProt P33480; glycoproteins F1 and F2, SwissProt P33481), West Nile virus (Genbank Acc. Nos. M12294 and M10103), herpes simplex virus (see above), Russian Far East encephalitis, Newcastle disease virus (see above), Venezuelan equine encephalomyelitis (Genbank Acc. No. L044599), rabies (Genbank Acc. No. U11736 and others), vaccinia (EMBL accession X91135) and varicella (GenPept U25806; Russell, 1994, Eur. J. Cancer, 30A, 1165-1171).

In addition to virally-derived FMGs used in the form normally present in the virus, viral FMG used in the invention may be engineered or modified to optimize its characteristics for therapeutic use (e.g. enhanced fusogenic activity, introduction of novel binding specificities or protease-dependencies to assist in targeting of the hybrid cell) as disclosed below.

Modifications Leading to Enhanced Fusogenicity

Truncation of the cytoplasmic domains of a number of retroviral and herpesvirus glycoproteins has been shown to increase their fusion activity, sometimes with a simultaneous reduction in the efficiency with which they are incorporated into virions (Rein et al, J. Virol. 1994, 68. 1773-1781; Brody et al, J. Virol. 1994, 68, 4620-4627; Mulligan et al, J. Virol. 1992, 66, 3971-3975; Pique et al, J. Virol. 1993, 67, 557-561; Baghian et al, J. Virol. 1993, 67, 2396-2401; Gage et al, J. Virol. 1993, 67, 2191-2201). Further, transmembrane domain swapping experiments between MLV and HTLV-1 have shown that envelopes which are readily fusogenic in cell-to-cell assays and also efficiently incorporated into virions may not necessarily confer virus-to-cell fusogenicity (Denesvre et al., J. Virol. 1996, 70, 4380-4386).

Modifications to Membrane Glycoproteins to Obtain Enhanced Selectivity of Syncytium Induction

The selectivity of syncytium induction by a viral FMG may be modified if so desired by fusing targeting moieties to the FMG that provide novel binding specificities. Such fusion proteins (i.e., a viral FMG fused to another protein) are described in U.S. Pat. No. 5,723,287, issued Mar. 3, 1998, hereby incorporated by reference. Novel binding specificities may be introduced into the FMG such that the modified FMG may recognize a selected receptor or antigen on a target cell, and thereby target the fusogenic activity to a specific cell type that expresses the targeted receptor or antigen. The altered glycoprotein may be tissue selective, as any tissue may give rise to a malignancy. Possible target antigens are preferentially expressed on breast, prostate, colon, ovary, testis, lung, stomach, pancreas, liver, thyroid, haemopoietic progenitor, T cells, B cells, muscle, nerve, etc. Additional possible target antigens include true tumor-specific antigens and oncofetal antigens. For example, B lymphocytes are known to give rise to at least 20 different types of haematological malignancy, with potential target molecules including CD10, CD19, CD20, CD21, CD22, CD38, CD40, CD52, surface IgM, surface IgD, idiotypic determinants on the surface of Ig, MHC class II, receptors for IL2, IL4, IL5, IL6, etc. Fusogenic membrane glycoproteins may be modified so as to contain receptor binding components of any ligand, for example, including monoclonal antibodies, naturally occurring growth factors such as interleukins, cytokines, chemokines, adhesins, integrins, neuropeptides, and non-natural peptides selected from phage libraries, and peptide toxins such as conotoxins, and agatoxins.

2. Non-Viral Fusogenic Membrane Proteins.

Cell-cell fusion occurs between some mammalian cells without the influence of viral membrane glycoproteins. For example, sperm and egg fusion occurs at fertilization. The fusogenic membrane protein carried by sperm has been identified as fertilin β, and the egg cell surface receptor is alpha-6, beta-1 integrin (Chen & Sampson, 1999, Chem. Biol. 6: 1-10).

Other examples of cell fusion occurring in mammalian systems include the fusion of myoblasts in skeletal and cardiac muscle, which function as viable syncytia. Futher, in the early stages of pregnancy, blastocyst attachment to the uterus involves the adhesion of the trophoblast to the uterine epithelial surface. Fusion between adjacent epithelial cells precedes the initial attachment of the blastocyst, and is followed by fusion between the trophoblast and the epithelium. A member of the cellular metalloproteinase/disintegrin family, MDC9, has integrin-binding, metalloproteinase and fusogenic functions and has been implicated in epithelial cell fusion that precedes trophoblast fusion. Also during pregnancy, the trophoblast, supporting the main functions of the placenta, develops from the fusion of cytotrophoblastic cells into a syncytiotrophoblast. The fusion of cytotrophoblastic cells is complex, and involves factors and pathways common to regulation of the apoptotic cascade, such as Bcl-2, Mcl-1 and topoisomerase IIα (Huppertz et al., 1998, Histochem Cell. Biol. 110: 495-508), as well as cAMP-dependent protein kinase type IIα (Keryer et al., 1998, J. Cell Sci. 111: 995-1004).

It is comtemplated that the cell fusion-promoting activities of proteins involved in sperm-egg fusion, myoblast fusion and cytotrophoblast syncytial formation can be exploited in the cell fusion methods of the invention.

3. Expression of FMPs and/or FMP Receptors.

Nucleic acid encoding an FMP or FMP receptor may be introduced to tumor cells or APCs in a recombinant expression vector. A wide array of mammalian expression vectors is available in the art. Mammalian expression vectors will comprise an origin of replication, a suitable promoter and optional enhancer, and also any necessary ribosome binding sites, a polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites, are frequently used to provide the required nontranscribed genetic elements.

A wide array of eukaryotic expression vectors are known in the art, including, for example, pWLneo, pSV2cat, pOG44, pXT1, pSG (Stratagene) pSVK3, pBPV, pMSG, pSVL (Pharmacia). However, any other plasmid or vector may be used as long as it is replicable and viable in the host (i.e., a mammal, preferably a human).

Promoter regions can be selected from any characterized gene as appropriate and may be incorporated into selected vectors using techniques well known in the art. Eukaryotic promoters include, for example, CMV immediate early, HSV thymidine kinase, early and late SV40, LTRs from retrovirus, and mouse metallothionein-I. Selection of the appropriate vector and promoter is well within the level of ordinary skill in the art. FMG expression is achieved, for example, by transfecting with an FMG expression construct regulated by or sensitive to, for example, tetracycline, rapamycin, IPTG, steriod hormone or other suitable small molecule inducer. An extensive list of cis-acting control elements exhibiting tissue-specific regulation is provided in the review of regulatable vectors for genetic therapy by Miller & Whelan (1997, Human Gene Ther. 8: 803-815). Further tissue-specific regulatory sequences are described by Miller & Vile (1995, FASEB J. 9: 190-199). Selection of the appropriate vector and promoter is well within the level of ordinary skill in the art.

4. Introduction of FMP and/or FMP Receptor Coding Sequences to a Cell.

A construct or constructs encoding an FMP as described above may be introduced to either a tumor cell or an APC, depending upon the exact embodiment of the invention, using any appropriate technique known in the art for introducing nucleic acids to cells. Among the methods well known in the art are transfection (e.g., by calcium phosphate precipitation, electroporation, DEAE-dextran, or liposomes) or transduction via viral infection (e.g., with any of a number of viruses adapted for use as vectors).

B. Antigen Presenting Cells Useful According to the Invention

Several different cell types qualify as APCs useful according to the invention. For the purposes of the present specification, a “professional APC” is a cell which has the cellular mechanisms necessary to present antigen in combination with MHC Class II antigens (that is, the APC can ingest antigen in a receptor-mediated manner, process it intracellularly, and re-present peptides derived from the antigen in combination with MHC Class I or II antigens). Naturally occurring professional APCs include macrophages, dendritic cells, and B lymphocytes. The surface markers associated with professional APCs include MHC Classes I and II, F_{c} receptor, and adhesion molecules.

MHC Class I markers include HLA-A, -B, and -C, and MHC Class II markers include HLA-DR, -DQ, and -DP. Fc receptors include, but are not limited to CD16 (FcRIII), CD23 (FcεRIIb), CDw32 (FcRII), and CD64 (FcRI). Adhesion molecules broadly expressed by antigen presenting cells include, but are not limited to CD11a (LFA-1α), CD18 (LFA-1β), CD29 (VLA-β), CD54 (ICAM-1), CD58 (LFA-3) and CD147 (neurothelin).

As noted above, the markers CD11a, -b, and -c, CD16, CD17, CD63, CD64, CD68 and CD71 expressed in combination define a macrophage. Macrophages may also express additional cell surface markers, e.g., one or more of: CD18, CD23, CD25, CD26, CD29, CD32, CD54, CD55, CD58, CD69, CD74, CD87, CD88, CD89, CD105, CD115, CD118, CD119, CDw121b, CD153, CD155 and/or CD163.

Dendritic cells are defined by the expression of CMRF-44, CD83 and CMRF-56 in combination, but may also express one or more of CD1a, -b, and -c, CD29, CD54, CD55, CD58, CD59, CD83, CD86, CD101, CD118 and/or CD148 among others.

B lymphocytes are defined by the expression of CD19, CD20, CD21, CD22, CD40, CD72, and CD78, but can also express one or more of CD5, CD6, CD10, CD23, CD24, CD25, CD26, CD29, CD30, CD31, CD32, CD35, CD37, CD39, CD45Rb, CD49d, -c, -d, and -e, CD54, CD55, CD58, CD59, CD69, CD70, CD71, CD73, CD74, CD75, CD76, CD78, CD79α, CD79β, CD80, CD81, CD82, CD83, CDw84, CD85, CD86, CD89, CD97, CD98, CD99, CD102, CD103, CDw108, CD118, CD119, Cdw121b, CD122, CD124, CD125, CD126, CD130, CD132, CDw137, CD138 and/or CD139, among others.

The professional APC used to form the hybrid cell comprises naturally occurring APCs, of which macrophages and dendritic cells are preferred. These cells may be obtained from the patient to whom the hybrid cell is to be administered, so that the APC used to make the fusion are autologous with respect to the patient. Desirably the APCs may be isolated from a sample of the patient's peripheral blood, using standard techniques.

For example, dendritic cells are isolated from bone marrow. Bone marrow samples are cultured in RPMI 1640 medium supplemented with 10% fetal calf serum, 5×10⁻⁵ M β-mercaptoethanol (βME) and 10% X63 GM-CSF producing cell supernatant (Melcher et al., 1999, Cancer Res., 59: 2802-2805; Inaba et al., 1993, J. Exp. Med. 178: 479-488). The medium is changed on day 3 of culture, and on days 7-9, adherent cells are dislodged by vigorous pipetting and all cells are collected. With GM-CSF as the only cytokine, dendritic cells are the predominant cell type to survive.

Dendritic cells may also be isolated from spleen tissue, if necessary or desirable over isolation from bone marrow, using the method described by Cao et al. (1999, Immunology 97: 616-625).

Macrophages may be isolated from peripheral blood, or, alternatively, from alveolar lavage fluid or from peritoneal lavage fluid. Macrophages are cultured in DMEM with 23 mM glucose, 2% FCS, 25 mM HEPES, sodium bicarbonate and gentamicin.

B cells are isolated according to the method of Suzuki and Sakane, 1989, J. Clin. Invest. 83: 937-944. Briefly, peripheral blood mononuclear cells are separated into T cells and non-T cells by means of a sheep red blood cell-rosette technique. B cells are obtained by further depletion of T cells remaining in the non-T cell fraction by complement-mediated cell lysis with OKT3 MAb. This is followed by depletion of monocytes by removal of cells adhering to petri dishes and by complement-mediated cell lysis with OKM1 MAb (both antibodies are available from Ortho Pharmaceutical Corp., Raritan, N..J.). The resultant B cell population is greater than 90% cells reactive with anti-Leu 16 MAb, no cells reactive with anti-Leu4 MAb (Becton Dickinson, Monolconal Center Inc., Mountain View, Calif.), <0.2% cells reactive with OKM5 MAb (Ortho), and <1% cells reactive with OKNK MAb (Ortho).

Primary dendritic cells, macrophages or B cells are transfected with an FMP encoding vector either by standard transfection methods (e.g., lipofection, DEAE dextran, electroporation, calcium phosphate) or by infection with the recombinant vector in a suitable medium, e.g, RPMI 1640 medium supplemented with 10% heat-inactivated FCS, 2 mM glutamine, 5×10⁻⁵ M βME, 100 U/ml penicillin and 100 U/ml streptomycin for 24-48 hours, whereupon expression of the vector-encoded FMG will be detectable on the APC surface.

B cells may also be cultured in DMEM, 10% FCS, 2 mM glutamine, 5×10⁻⁵ M βME, 100 U/ml penicillin and 100 U/ml streptomycin.

As an alternative to isolating APCs from the patient, APCs may be obtained from another individual who shares the same MHC Class I and II antigens with the patient.

A further option is to produce “synthetic” APCs, by introducing into a cell that is not an antigen presenting cell the gene or genes encoding those parts of the APC machinery which are absent in order to confer APC activity on the cell (e.g. introducing genes encoding relevant MHC Class II antigens, and/or genes encoding F_{c} receptors and adhesion molecules as described above). As an example, primary fibroblasts (isolated, for example, from a skin biopsy), may be transfected by lipofection or infection with a recombinant virus carrying sequences coding for HLA-DR, DQ or DP. Co-transfection with sequences coding for cell adhesion molecules such as ICAM-1 or VLA-β makes the synthetic APC yet more efficient at antigen presentation and stimulation of the immune response.

C. Tumor Cells and Cell Markers Useful According to the Invention

The tumor cells used to form the hybrid cell may be tumor cells taken from the patient (autologous tumor cells), or they may be exogenous tumor cell vaccines (e.g. see Hoon et al, J Immunol 1995 154; 730-737) which may be MHC-matched, partially MHC-matched, or MHC mismatched with the patient. Methods of culturing tumor cells are well known in the art. Tumor cells are dissociated from resected tumor tissue using, for example, pronase or collagenase digestion. The medium and growth factor requirements will vary with the particular tumor cell type being cultured, however a reasonable starting point for medium is a standard DMEM or RPMI medium supplemented with fetal calf serum (1 to 20%) and antibiotics. One skilled in the art can select the appropriate growth factor and nutrient supplements to maintain and grow the tumor cells in culture.

Tumor antigens include, but are not limited to, prostate specific antigen (PSA; Osterling, 1991, J. Urol., 145: 907-923), epithelial membrane antigen (multiple epithelial carcinomas; Pinkus et al., 1986, Am. J. Clin. Pathol. 85: 269-277), CYFRA 21-1 (lung cancer; Lai et al., 1999, Jpn. J. Clin. Oncol. 29: 421-421) and Ep-CAM (pan-carcinoma; Chaubal et al., 1999, Anticancer Res. 19: 2237-2242).

Oncofetal tumor antigens alphafetoprotein and carcinoembryonic antigen are usually only highly expressed in developing embryos, but are frequently highly expressed by tumors of the liver and colon, respectively, in adults. Other oncofetal tumor antigens include, but are not limited to, placental alkaline phosphatase (Deonarain et al., 1997, Protein Eng. 10: 89-98; Travers & Bodmer, 1984, Int. J. Cancer 33: 633-641), sialyl-Lewis X (adenocarcinoma, Wittig et al., 1996, Int. J. Cancer 67: 80-85), CA-125 and CA-19 (gastrointestinal , hepatic, and gynecological tumors; Pitkanen et al., 1994, Pediatr. Res. 35: 205-208), TAG-72 (colorectal tumors; Gaudagni et al., 1996, Anticancer Res. 16: 2141-2148), epithelial glycoprotein 2 (pan-carcinoma expression; Roovers et al., 1998, Br. J. Cancer. 78: 1407-1416), pancreatic oncofetal antigen (Kithier et al., 1992, Tumor Biol. 13: 343-351), 5T4 (gastric carcinoma; Starzynska et al., 1998, Eur. J. Gastroenterol. Hepatol. 10: 479-484,; alphafetoprotein receptor (multiple tumor types, particularly mammary tumors; Moro et al., 1993, Tumour Biol. 14: 11-130), and M2A (germ cell neoplasia; Marks et al., 1999, Brit. J. Cancer 80: 569-578).

D. How to Make Hybrid Cells According to the Invention

Formation of the hybrid cell is brought about by expression of an FMP on one of the fusion partners. The corresponding FMP receptor, or a receptor for a modified FMP fusion protein (that is selective for a partner cell surface receptor), on the one or more other cell fusion partners. Generally, neither the tumor cell nor the APC will naturally express the FMP, so it will normally be necessary to introduce nucleic acid encoding the FMP into at least one of the fusion partners: the tumor cell and/or the APC may be engineered in this way, using conventional techniques for introducing nucleic acid into cells as described above and known in the art. It may, depending upon the cell types and type of FMP employed, also be necessary to engineer one or both fusion partners so as to express the relevant FMP receptor, but it will be apparent to those skilled in the art that this can often be avoided by appropriate selection of FMP and cell types employed in the formation of the hybrid cell. That is, one may select an appropriate FMP or FMP fusion protein containing a ligand for a cell surface receptor based upon the endogenous expression of an FMP or other cell surface receptor on the target cell. Generally, for a viral FMG, a cell type known to be infected by the virus from which the FMG is derived would be expected to express the appropriate FMG receptor.

It is preferred that expression of the FMP, and/or its reciprocal receptor in the fusion partner cells, is regulatable so as to prevent premature fusion (i.e., fusion of cells before mixing with partner cells). Regulatable expression is achieved using, for example, expression systems that are drug inducible (e.g., tetracycline, rapamycin or hormone-inducible). Heat-sensitive regulation of expression may also be used to minimize the fusion of cells until they are mixed with the desired fusion partner. For example, transcription regulated by a co-transfected, temperature sensitive transcription factor active only at 37° C. is used if cells are first grown at, for example, 32° C., and then switched to 37° C. or introduced to a patient (normal body temperature is 37° C.) when fusion is desired.

Viral Vectors

Viral vectors that can be used to deliver foreign nucleic acid into cells include but are not limited to retroviral vectors, adenoviral vectors, adeno-associated viral vectors, herpesviral vectors, and Semliki forest viral (alphaviral) vectors. Defective retroviruses are well characterized for use in gene transfer for gene therapy purposes (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). An AAV vector such as that described in Tratschin et al. (1985) Mol. Cell. Biol. 5:3251-3260 can be used to introduce nucleic acid into cells. A variety of nucleic acids have been introduced into different cell types using AAV vectors (see for example Hermonat et al. (1984) Proc. Natl. Acad. Sci. USA 81:6466-6470; and Tratschin et al. (1985) Mol. Cell. Biol. 4:2072-2081).

Measles virus F and H Glycoprotein Vectors and Transduction

When expressed concurrently in the same cell, measles virus F and H glycoproteins can mediate cell-cell fusion with neighboring cells, provided the neighboring cells express the measles virus receptor (CD46). Human cells express the CD46 measles virus receptor, whereas murine cells do not. A retroviral vector capable of transferring the measles virus F and H genes may be used to demonstrate the therapeutic potential of gene therapy for cancer therapy, as described herein. The vectors are used to direct expression of the fusogenic membrane protein (FMP) in a cell as described herein.

1. Construction of retroviral vector plasmid coding for measles virus F and H glycoproteins is described in detail in WO98/40492, hereby incorporated by reference. Briefly, a plasmid is constructed using standard cloning methods. The plasmid, from left to right (representing 5′ to 3′ on a genetic map, contains an LTR (Moloney murine leukaemia virus long terminal repeat), a Moloney murine leukaemia virus packaging signal, an IRES (poliovirus internal ribosome entry site), a measles virus H glycoprotein coding sequence, a measles virus F glycoprotein coding sequence, and a phleomycin resistance marker. The vector backbone is either pUC or pBR322—based. The coding sequence of the measles virus H gene is cloned from pCGH5 (Cathomen et al, 1995, Virology, 214, 628-632), into the NotI site of the retroviral vector plasmid pGCP (which contains the poliovirus internal ribosome entry site flanked by NotI and ClaI cloning sites). The measles virus F gene is then cloned from pCGF (Cathomen et al, 1995, Virology, 214, 628-632) into the ClaI site of the same vector, 5′ of the internal ribosome entry site to produce the vector named pHF. A phleomycin selectable marker gene is then introduced into the vector 5′ of the 5′ LTR.

2. Preparation of Retroviral Vector Stocks.

The plasmid pHF is transfected into amphotropic retroviral packaging cell lines which were derived from murine fibroblasts. Suitable packaging cell lines are widely available and include the NIH3T3-derived cell lines PA317 and GP+env AM12. Stably transfected packaging cells are selected in phleomycin 50 ug/ml and used as a source of HF retroviral vectors capable of efficiently transferring the measles virus F and H genes to human and murine target cells.

Monitoring the Formation of Hybrids

The formation of hybrids is monitored in several ways. First, microscopic analysis is useful. A cell sample is taken from an in vitro cell fusion culture, or a cell sample is obtained from an appropriate site (e.g., a site where a tumor is located) in an individual to which an APC expressing an FMP has been administered (24-48 hours after such administration). Depending upon the cell types fused, the actual size of fused cells may be larger than either fusion partner alone. In addition, only those cells that have fused express cell surface markers of both the fusion donor and the fusion target cell. Therefore, simultaneous staining of hybrid cells with differentially-labeled antibodies or lectins specific for cell surface markers on each cell type reveals hybrid cells as the only cells to simultaneously express markers for both cell types. Antibodies or lectins to cell surface markers may be differentially labeled, for example, with the fluorescent markers fluorescein and rhodamine.

Another way of monitoring hybrid cell formation is by fluorescence activated cell sorting (FACS). The mixture of APCs and tumor cells is reacted with two differentially-labeled fluorescent antibodies, one specific for a cell surface marker that is expressed by the APCs, and the other antibody specific for a cell surface marker expressed only by the tumor cell. Cells exhibiting a fluorescence spectrum indicating the presence of both labels are then sorted from those exhibiting only one of the two labels by a FACS apparatus. Markers specific for APCs are described herein above and known in the art. For example, the following antibodies are available for the stated cell and antigenic types: B-cell antibodies: RA3-3A1/6.1, Cy34.1.2, 10-1.D.2; Macrophage antibodies: 10B9, 14E5, 3C10; Dendritic cell antibodies: 1.G; Breast cancer antibodies: 3B18, 33F8; Bladder cancer antibodies: ME 195, MF116; Cancer cells, human: Ch13, De8, PO71; Colon adenocarcinoma: CLT 85, CLH 6; Colorectal carcinoma: XMMCO-791, 1116-NS-19-9; Lung carcinoma: L3, Ri37; Melanoma: LI27, E 20; Ovarian carcinoma: MF 116, OVB-3; and Vulva carcinoma: VLN1F9, VLN5C7. Antibodies specific for many such markers are commercially available in fluorescently labeled form, and methods of labeling antibodies are also well known, if necessary. Useful markers for staining tumor cell fusion partners include tumor antigens specific for the given tumor cell type, as well as non-tumor antigens expressed by the tumor cell that are not also expressed by the APC. Antibodies specific for tumor antigens are widely available commercially.

A number of different cell fusion donor/acceptor cell partnerships can be envisaged in which expression of one or more FMPs allows fusion of hybrids useful for killing tumor cells. In one approach, the tumor cell-APC hybrids are created ex vivo and then introduced to the patient. In another approach, tumor or APCs are engineered to express one or more FMPs and then introduced to a patient to allow the formation of tumor-APC hybrids in vivo. These approaches and their application to several different cell/cell partnerships or combinations are described in the following examples.

### EXAMPLE I

Ex Vivo Manipulations followed by in Vivo Vaccination with FMG-derived Hybrids:

Autologous tumor cells recovered from the patient at surgery are engineered to express one or more FMPs (using the rapid adenoviral transfer protocol of Diaz et al. (Gene Therapy 1998 5:869-879) and then mixed ex vivo with patient dendritic cells or macrophages (readily prepared from peripheral blood by means of conventional techniques known to those skilled in the art). The resulting hybrid cells are then able to process and present antigens, including the autologous tumor antigens expressed by the tumor cells, as complexes with MHC II molecules. The number of cells (APC and tumor cell) used for fusion depends upon the tumor type and its size, as well as upon the chosen mode of administration (see below). Generally, from 1×10⁶ to 1×10¹⁰ total cells are used to make cell hybrids. The ratio of APC to tumor cell may be, for example 100:1, 0:1, 1:1, 1:10, or 1:100, depending upon the efficiency of hybrid formation between the particular APC and tumor cells.

Autologous tumor cells (10⁶) from a patient are resuspended in RPMI 1640 medium with 10% FCS, as described above, and mixed with recombinant retroviral vector, containing a coding sequence for F and H proteins of measles virus, at a multiplicity of infection of 3, and incubated at 37° C. for 24 hours. The cells are then washed and resuspended in the same medium. Trandsuced tumor cells are then incubated 1:1 (10⁶ cells:10⁶ cells) with dendritic cells obtained from the same patient at 37° C. in the same medium for 48 to 72 hours, or until hybrids form.

Alternatively, 10⁶ dendritic cells, macrophages or B lymphocytes, isolated from a patient as described above, are resuspended in RPMI with 10% FCS, mixed with recombinant retroviral vector encoding MMLV env at a multiplicity of infection of 3, and incubated at 37° C. for 24 hours. Transduced cells are then washed and resuspended in the same medium, mixed with 10⁶ autologous tumor cells from a patient, and incubated at 37° C. for 48 to 72 hours to permit cell-cell fusion.

There are several different ways to isolate hybrid cells from the culture prior to administering them to a patient. First, hybrid cells may be separated from non-fused cells by FACS. Alternatively, physical methods that take advantage of size or bouyant density differences between hybrids and both non-fused cell types are effective. Examples of methods that separate cells by size or bouyant density include centrifugal elutriation (Chang et al., 1999, Biol. Blood Marrow Transplant 5: 328-335) and sedimentation through a gradient (e.g., Percoll; Lichtenberger et al., 1999, J. Immunol. Meth. 227: 75-84). Optimally, the hybrid cells are isolated by a combination of the physical and the cytochemical methods, for example, by separating on a Percoll gradient, then cell sorting by differential labeling. These hybrids also present the FMP-derived immunogenic peptides which enhance the adjuvant quality of the hybrids (Nabel et al. Hum. Gene Ther. 1992 3:399-410; Vile and Chong, Cancer and Metastasis Rev. 1996 15:351-364) through cross-priming of host APCs with tumor-derived antigens released following killing of the immunogenic target hybrids (Huang et al. Science 1994 264:961-965). The fusion hybrid cells may have an added adjuvant effect through the expression of immunogenic FMG epitopes.

### EXAMPLE II

Allogeneic tumor cells (10⁶) (which are already available for certain tumor types (Hoon et al. Journal of Immunology 1995 154:730-737) are resuspended in RPMI/10% FCS medium and transfected with recombinant retroviral expression vector encoding measles virus F and H glycoproteins at a multiplicity of infection of 3. If desired, the vector contains an inducible promoter which controls F and H glycoprotein expression, for example the tetracycline regulatable promoter (Gossen & Bujard, 1995, Science 268: 1766-1769). Inducible FMP expression prevents cells from fusing prior to the addition of APC fusion partners. The FMP-engineered allogeneic tumor cells are then mixed with 10⁶ freshly prepared patient dendritic cells and/or macrophages ex vivo and incubated in the same medium at 37° C. for 48 hours in the presence of 30 μg/ml tetracycline, which induces FMP expression in order to generate an allogeneic tumor cell-APC hybrid. Without intending to be limited by theory, the APC component of the hybrid vaccine is believed to enhance the presentation of the shared antigens between the allo-cells and the patient's tumor.

Alternatively, 10⁶ freshly prepared dendritic cells or macrophages are transduced with a retroviral vector encoding measles virus F and H proteins at a multiplicity of infection of 3 in RPMI/10% FCS medium for 24 hours at 37° C., washed and resuspended in the same medium prior to contacting the cells with 106 allogeneic tumor cells for 48-72 hours under FMP expression inducing conditions.

### EXAMPLE III

Allogeneic tumor cells (10⁶), which are known to be MHC-matched with the patient for relevant antigen presenting loci (Boon & van der Bruggen, Journal of Experimental Medicine 1996 183:725-729), are transfected with the recombinant vector engineered to express the FMG inducibly (as described in Example II) and are then mixed with freshly resected patient tumor cells. The hybrid cells provide a potent adjuvant effect (with a combination of partially allo-MHC expressed along with FMG-derived epitopes) but also express tumor derived antigens from both the allo-partners (shared antigens) and the autologous partner cells. These tumor associated antigens are presented through the MHC molecules provided by the fusion allo-donor cells.

### EXAMPLE IV

Direct in Vivo Delivery of FMG-expressing Donor Cells

Allogeneic tumor cells (10⁶) (either MHC-mismatched or partially MHC-matched with the patient), or even autologous cells are transfected with a vector engineered to express MMLV env and then 106 cells are delivered directly to local tumor deposits (either by intratumoral injection or by injection into appropriate body cavities). 5 days later, a tumor biopsy may be performed and the presence of large multinucleate cells which stain for botha tumor antigen and an APC antigen will indicate syncytium formation. The FMP-expressing allogeneic line of the same histological type as the tumor thus is indicated to traffic within the tumor deposits and fuse with the tumor cells, thereby promoting killing of the tumor cells. After several days to several weeks, a reduction in diesaes symptoms may occur.

### EXAMPLE V

Autologous dendritic cells and/or macrophages (10⁶) are transfected with a vector containing sequences encoding FMLV SU glycoprotein having a trp to gly substitution at amino acid 120. Transfection is performed in RPMI/10% FCS at 37° C. for 24 hours, and cells are then washed and resuspended in isotonic buffer and then directly injected into established tumor deposits. After 24-48 hours, fusion of the transfected dendritic cells or macrophages with the patient tumor cells generates hybrids which present both autologous tumor cell antigens and MHC class II molecules on their surface. The resulting presentation of tumor cell antigens results in a potent cytotoxic T cell response to cells expressing tumor cell antigens.

Whether using the ex vivo or in vivo approach to generating hybrid cells, the hybrid cells have several identifying characteristics. Hybrid cells express tumor antigens (autologous or allo-antigens) expressed by the tumor cells, MHC class II molecules expressed by the APC, and the FMP.

Whether hybrid cells are administered to or formed within a patient, the fusion of tumor cells via the FMG leads to direct killing of tumor cells. This activity is evidenced by shrinkage of the tumor (for solid tumors) or a decrease in the number of tumor cells in an appropriate fluid sample (for non-solid tumors; e.g., blood or bone marrow). In addition, hybrid cells expressing tumor-derived peptide antigens in complexes with MHC class II molecules provided by the APC stimulates an anti-tumor immune response, as indicated by at least a 5-fold expansion of tumor-specific T cells (in comparison to T cells in an untreated patient).

The FMPs, particularly the virally-derived FMGs, are themselves highly immunogenic. This property has several beneficial consequences in the methods of the invention. First, as described above, the hybrid cells provide a potent in vivo adjuvant effect: the combination of an allo-MHC, along with immunogenic FMG-derived epitopes is likely to generate an effective in vivo immune stimulatory effect which may resemble a so-called “Danger” signal which has been suggested to be important for the generation of effective immunity (Matzinger, Annual Review of Immunology 1994 12:991-1045) and for breaking tolerance to self tumor antigens (Melcher et al Nature Medicine 1998 4:581-587). Second, the lifetime of the hybrid cells themselves is limited by the immune response to cells expressing the FMG. Not only does this improve upon the safety of tumor cell vaccines, but immune-mediated lysis of the hybrid cell also increases the turnover and availability of the tumor-associated antigens to the patient's immune system in general, and to the patient's antigen-presenting cells in particular.

Therefore, in addition to looking for direct killing of the tumor by FMG-mediated syncytia formation, one also looks for the presence or expansion of activated T cells specific for tumor cell antigens expressed by the tumor being treated.

### EXAMPLE VI

Fusion Hybrids May be Used to Deliver a Therapeutic Substance to Cells.

For the delivery of a therapeutic substance to cells, fusion hybrids described herein may be transfected to contain a therapeutic substance, such as a therapeutic gene. Alternatively, where fusion occurs in vivo, within the subject being treated. Delivery of fusion hybrids, diluted in a physiologically acceptable diluent, is accomplished by the same methods of delivery and in similar amounts as used to introduce cells for treatment of malignant disease. It is contemplated according to the invention that a fusion hybrid may be used not only to induce syncytium formation in the patient who is being treated, but the hybrid also may be transduced so as to contain a gene encoding a cytotoxic agent, such as nitroreductase, such that fusion of the hybrid cell with cells in the body results in expression of nitroreductase in the syncytium, and faster killing of the cells, also permitting a bystander effect in the area of the syncytium. In this way, the effect of the fusion hybrid on cell killing via syncytium formation is increased.

Methods of the invention may be tested in an immunocompetent mouse tumor model, as follows. Primary mouse dendritic cells(DC) isolated by the method of Inaba et al. (1993, supra) are transduced with a retroviral vector encoding measles virus F and H fusogenic membrane glycoproteins (vector as in Russell et al., WO98/40492). For infection, 10⁶ cells are incubated with recombinant virus at a multiplicity of infection of 3 in DMEM/10% FCS with 4 mM glutamine for 24 hours. Infected cells are then washed and incubated in fresh medium.

Immunocompetent C57B1/6 mice are injected subcutaneously with 2×10⁶ tumor cells from the syngeneic colorectal tumor cell line CMT93, and tumors are allowed to grow to approximately 1.0 cm in diameter.

Hybrid cells are made by mixing measles virus F+H-expressing DCs with CMT93 cells (colorectal tumor cell line) at a ratio of 10:1 (e.g., 10⁶ F+H expressing DCs to 10⁵ CMT93 cells) and incubating at 37° C. in DMEM/10% FCS/4 mM glutamine, in a 5% CO₂ environment for 1 to 6 hours. Hybrid formation is evaluated microscopically with fluorescein-labeled anti-CD83 (dendritic cell marker) and rhodamine-labeled anti-CA19-9 (colorectal tumor marker; antibody available from Chemicon Inc., Temecula, Calif., catalog No. MAB4048) antibodies.

### EXAMPLE VII

Evaluation of Tumor-protective Effect.

The protective effect of hybrid cells is investigated by injecting hybrid cells (1×10⁵ to 2×10⁵) into animals not previously injected with tumor cells, followed one week later by sub-cutaneous injection of non-hybrid CMT93 cells. Tumor formation is compared with that observed in an animal injected with tumor cells but not previously inoculated with hybrid cells. Lack of tumor growth or slower tumor growth in the hybrid-injected animals indicates a protective effect of the hybrid cells.

### EXAMPLE VIII

Evaluation of Therapeutic Effect Against Established Tumors.

The therapeutic effect of hybrid cells on established tumors is investigated by injection of 1×10⁵ to 2×10⁵ hybrid cells directly into established tumor masses. Tumor size is monitored daily, and a slowing or cessation of growth relative to non-hybrid-injected animals or a decrease in the size of the tumor relative to the size at the commencement of treatment is indicative of a therapeutic effect against established tumors.

### DOSAGE, PHARMACEUTICAL FORMULATIONS AND MODE OF ADMINISTRATION

Hybrid cells of the invention may be provided as a therapeutic composition for the treatment of malignant disease.

The preparation of the therapeutic composition comprises the steps of preparing the hybrids and placing them in admixture with a physiologically acceptable diluent (for example, buffered saline). The concentration of hybrid cells in the preparation will vary, depending upon the chosen route of administration. For example, local (e.g., intratumor) administration requires higher concentrations of hybrid cells than systemic (e.g., intravenous) administration. For intratumor delivery, hybrid cells are suspended in an acceptable diluent at about 1×10^{6 to} 1×10⁸ cells per ml, and 0.2 to 5 ml of hybrid cell suspension are administered. For systemic delivery, hybrid cells are suspended in an acceptable diluent at about 1×10³ to 1×10⁷ cells per ml, and 10 ml to 1 liter of cell suspension is administered. Diluents include, for example, sterile, pyrogen-free phosphate-buffered saline, and Ringer's lactate solution.

Hybrids of tumor cells and APCs will generally be useful for treatment of tumors similar to or identical to (in the case of autologous tumor cells) that tumor cell type used to make the fusion. Generally, treatment comprises the steps of making the hybrids and introducing them to an individual in need of treatment.

Hybrid cells of the invention are administered to a patient in need of treatment by any of a number of routes as noted above. The selection of a particular route depends upon the manner in which the hybrid cells were formed.

In those cases in which hybrid cells are formed ex vivo, hybrid cells are administered as a suspension in a physiologically acceptable diluent. The suspension may be administered intravenously, intraperitoneally, or by direct injection into a tumor or into the vicinity of a tumor. The concentration of hybrid cells in the preparation given will vary, as noted above, depending upon the route of administration, and from about 2×10⁵ to about 5×10⁸ hybrid cells are administered at a time. Hybrid cells may be administered once, twice, three times or more, to treat a given malignant disease, with the frequency of administration depending upon measured therapeutic effect (see below).

In those cases in which hybrids are formed in vivo, or more specifically, in situ, autologous APCs or allogeneic tumor cell lines modified to express one or more FMPs may be directly injected into a tumor as a suspension in a physiologically-acceptable diluent. The numbers and concentrations of fusogenic cells administered are similar to the number of cell hybrids administered when such hybrids are administered (i.e., from about 2×10⁵ to about 1×10⁸ total cells in volume from about 0.2 ml to about 5 ml). These numbers can, of course, be modified if the efficiency of hybrid formation is known, for example, through in vitro measurement, to be low for a particular donor-target combination.

The efficacy of treatment of a tumor with any of the hybrids of the invention may be evaluated by monitoring the size of a tumor (in the case of solid tumors) or the number of tumor cells in a sample of a given size (for non-solid tumors). Tumor size may be monitored according to any of a number of means known in the art, including external palpation, ultrasound, magnetic resonance imaging, or through tumor imaging techniques specific to a given tumor type, such as illumination with a labeled tumor-antigen-specific antibody. Tumor growth is considered to be halted or arrested according to the invention if the size of a tumor or the number of tumor cells in a sample of a given size does not increase over time. A tumor is considered to be reduced in size or abundance of cancer cells if it is at least 10%, 20%, 30%, 50%, 75%, 90% smaller (or less abundant) or more, including 100% smaller (that is, the complete absence of tumor cells) than it was immediately prior to the commencement of treatment.

### OTHER EMBODIMENTS

Other embodiments are within the following claims:

1. An isolated cell expressing on its surface a fusogenic membrane protein and a professional antigen presenting cell marker.

2. The isolated cell of claim 1, further comprising on its surface a tumor cell marker.

3. The cell of claim 1, wherein said fusogenic membrane protein is a viral fusogenic membrane glycoprotein.

4. The cell of claim 1, comprising a tumor cell fused to a professional antigen presenting cell, wherein said tumor cell and/or said antigen presenting cell is obtained from a patient to whom the cell is to be administered.

5. The cell of claim 1, wherein said tumor antigen is an antigen which is expressed in a tumor cell line.

6. A method of making a hybrid cell, the method comprising
contacting a tumor cell with a professional antigen presenting cell under conditions which permit cell-cell fusion, wherein one of said tumor cell or said profesional antigen presenting cell expresses a fusogenic membrane protein receptor.

7. The method of claim 6, wherein said contacting step is performed in vitro or ex vivo.

8. The method of claim 6, wherein said contacting step is performed in situ in a patient.

9. A method of preparing a therapeutic composition for the treatment of malignant disease, the method comprising the step of admixing a cell of claim 1 or the cell of claim 2 with a physiologically acceptable carrier.

10. A therapeutic composition comprising a cell of claim 1 or claim 2, in admixture with a physiologically acceptable carrier.

11. A method of treating a malignant disease in a mammal, the method comprising the step of administering said cell of claim 2 to said mammal in an amount effective to reduce a symptom of said malignant disease.

12. The method of claim 11, further comprising, prior to said administering, the step of fusing a tumor cell with a professional antigen presenting cell in vitro or ex vivo to form the cell of claim 1.

13. A method of treating a malignant disease in a mammal, the method comprising the step of administering a professional antigen presenting cell expressing a fusogenic membrane protein to said mammal in an amount effective to reduce a symptom of the malignant disease.

14. A method of treating a malignant disease in a mammal, comprising administering to said mammal an autologous tumor cell suspension expressing on its surface a fusogenic membrane protein in an amount effective to reduce a symptom of said disease.

15. A method of vaccinating a mammal against a malignant disease, comprising administering to the mammal the cell of claim 2 in an amount effective to elicit an increase in the number of T cells specific for said tumor cell marker.

16. A method of vaccinating a mammal against a malignant disease, comprising administering to the mammal an autologous tumor cell expressing on its surface a fusogenic membrane protein and a tumor cell marker in an amount effective to elicit an increase in the number of T cells specific for said tumor cell marker.
